SEQUENCE LISTING (1 sequence)

SEQ ID NO: 1 - protein, 12 aa, Artificial Sequence
  a matrix metalloproteinase-sensitive peptide, abbreviated PQ
GGGPQGIWGQGK

(19) United States
(12) Patent Application Publication
(10) Pub. No.: US 2020/0061214 A1
(43) Pub. Date: Feb. 27, 2020

(54) Compositions for Real-Time Oxygen Measurements and Methods of Making and Using Same

(71) Applicant: Duke University, Durham, NC (US)

(72) Inventors: Mohamed Ibrahim, Durham, NC (US); John Wu, Durham, NC (US); Ryan Schweller, Durham, NC (US); Bruce Klitzman, Durham, NC (US)

(21) Appl. No.: 16/488,434

(22) PCT Filed: Feb. 23, 2018
(86) PCT No.: PCT/US2018/019536

§ 371 Date: Aug. 23, 2019

Related U.S. Application Data
(60) Provisional application No. 62/462,969, filed on Feb. 24, 2017.

(51) Int. Cl. A61K 49/00; A61B 5/00; A61B 5/1455

(52) U.S. Cl. CPC A61K 49/0036; A61B 5/0084; A61B 5/0071; A61K 49/0073; A61K 49/0054; A61K 49/0015; A61K 49/0091; A61B 5/14552

(57) ABSTRACT

The present disclosure provides compositions and methods for real-time oxygen measurements. More particularly, the present disclosure relates to oxygen-sensing compositions including a metalloporphyrin compound.

### CROSS-REFERENCE TO RELATED APPLICATIONS

This application claims the benefit of U.S. Provisional Patent Application No. 62/462,969, filed Feb. 24, 2017, all of which is incorporated herein by reference in its entirety.

### BACKGROUND OF THE INVENTION

### Field of the Invention

The present disclosure provides compositions and methods for real-time oxygen measurement. More particularly, the present disclosure relates to oxygen-sensing compositions including a metalloporphyrin compound.

### Description of the Related Art

The circulatory system employs specialized oxygen-carrying molecules in the blood to deliver oxygen from the lungs to other tissues throughout the body. To function normally, every organ in the body must contain sufficient amounts of oxygen in every tissue. Therefore, differing oxygen levels in tissue can be indicative of tissue structure abnormalities, diseases, or defects, whether caused externally or genetically. Therefore, reliable and accurate measurement of the oxygen supply in mammal tissue is important to ensure that the supply of oxygen is adequate and the tissue is healthy. Existing products and standard of care methods to measure oxygenation can be invasive, poorly localized, subject to positioning difficulties and motion artifact, time-consuming, and expensive.

The use of fiber optic probes employing luminescence quenching for such biomedical measurements has become very popular because these probes are easy to insert, involve no electrical hazards, and are economical to produce. In a fiber optic probe employing luminescence quenching, light from a suitable source is transmitted through long, thin, optically conducting flexible fibers of glass, plastic, or other transparent material to a receptor terminal containing a luminescent dye on an oxygen permeable support medium. The light causes the dye to luminesce and oxygen present in the blood or tissue quenches the luminescence. The light is then returned along the optical fiber to a light measuring instrument containing photomultiplier or photodiode tubes and an electronic computing circuit for processing.

While a number of fiber-optic probe devices have been reported, none of these devices are entirely satisfactory. Many luminescent dyes are fluorescent dyes and require expensive instrumentation because such dyes have short emission lifetimes and are not highly sensitive to quenching. Most fluorescent dyes are also sensitive to several anesthetic gases, often present in patients requiring tissue oxygenation monitoring. Furthermore, the permeability and solubility of the physiological gas in the support medium for the dye is not always optimal for the particular instrumentation employed.

Accordingly, there exist a need for non-invasive, real-time, and continuous methods for monitoring oxygenation at any given location or anatomical site that would be challenging for existing products.

### SUMMARY OF THE INVENTION

One aspect of the present invention includes an oxygen-sensing compound including a metalloporphyrin encapsulated within a polymer particle.

Another aspect of the disclosure provides an oxygen sensor composition including an oxygen-sensing compound embedded within a hydrogel carrier, wherein the oxygen-sensing compound includes a metalloporphyrin encapsulated within a polymer particle.

Another aspect of the disclosure provides an optical fiber device for the detection of oxygen in a deep body organ of a subject comprising: (i) an optic probe that is coated with an oxygen sensor composition of the disclosure; (ii) an optical fiber in electrical communication with the optic probe; and (iii) a remote detector in electrical communication with the fiber.

Another aspect of the disclosure provides a method for monitoring oxygenation (e.g., oxygen concentration and/or oxygen tension) in a subject, the method including:
(i) administering to a subject a therapeutically effective amount of an oxygen sensor composition of the disclosure;
(ii) activating an excitation light source to excite the oxygen-sensing compound;
(iii) measuring the fluorescence or phosphorescence from the oxygen-sensing compound; and
(iv) calculating the concentration of oxygen from the measurement.

Another aspect of the disclosure provides a method for monitoring oxygenation (e.g., oxygen concentration and/or oxygen tension) in a subject, the method including:
(i) coating a tip of an optic probe with an oxygen sensor composition of the disclosure;
(ii) inserting the optic probe into tissue of the subject;
(iii) activating an excitation light source to excite the oxygen-sensing compound;
(iv) measuring the fluorescence or phosphorescence from the oxygen-sensing compound; and
(v) calculating the concentration of oxygen from the measurement.

Another aspect of the disclosure provides a system for monitoring oxygenation (e.g., oxygen concentration and/or oxygen tension), the system comprising (i) an oxygen sensor composition of the disclosure; (ii) an excitation light source; and (iii) an instrument for measuring and reporting fluorescence or phosphorescence from the activated oxygen-sensing compound.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 illustrates spectroscopic characterization of the oxygen sensor composition prepared as provided in Example 2. Combining the oxygen-sensitive porphyrins with polystyrene leads to the formation of microparticles with spectroscopic properties which are different from the original oxygen-sensing porphyrin. A. The absorbance spectra of the metalloporphyrin before combining with polystyrene. Difference in absorbance is observed between the diluted and the concentrated compound, the peak of the absorption is at ˜680 nm wavelength. B. The absorbance spectra after combining with polystyrene. The compound demonstrated stable intensity in diluted and concentrated states. The compound demonstrated shift in the absorbance spectra into 2 distinct peaks at ˜440 nm and ˜640 nm wavelength. C. The emission spectrum of the compound without polystyrene, excited by ˜640 nm wavelength light. D. The ambient and deoxygenated emission spectra of the compound with polystyrene excited by ˜640 nm spectra and shifted to emit at ˜810 nm wavelength. This increase in the emission peak improves collection of the emitted signal through deeper implantation depth.

FIG. 2 illustrates the lifetime of fluorescence decay characterization of the oxygen sensor composition as provided in Example 2. A. The compound without polystyrene demonstrated very short lifetime of fluorescence decay less than 10 nanoseconds. B. The compound after combining with polystyrene demonstrated significant increase in the lifetime of fluorescence decay from nanoseconds to microseconds, which increases the range of detecting changes in oxygenation and hence increases the possibility of using the microparticles for the use in physiologic and pathologic conditions. C. illustrates fluorescent imaging characterization of the oxygen sensor composition.

FIG. 3 illustrates the responsiveness and sensitivity of the oxygen sensor composition as provided in Example 2.

FIG. 4 illustrates the particles of Pd(II) tetraphenyltetrabenzoporphyrin (PdTPTBP) encapsulated with polystyrene. The particles had average mean particle size of 2.5 μm.

FIG. 5 illustrates the real-life oxygen tension levels in of a sensor of the disclosure implanted in the myocardium of a perfused ex vivo swine heart.

FIG. 6 illustrates the fluorescence and real-life oxygen tension levels when the sensors of the disclosure are implanted intradermally and subcutaneously in ex vivo swine skin. A. Absolute fluorescence imaging of the implanted sensor, Intradermal (right sensor), subcutaneous at depth of 5 mm (middle sensor) and subcutaneous at depth of 7 mm (left sensor). B. Oxygen tension readings using real-time fluorescence lifetime decay from the deep subcutaneous sensor. Swine skin was placed in saline solution, and 100% O₂ was bubbled for 24 minutes, followed by CO₂ bubbling for 9 minutes. The oxygen sensor has appropriately responded to changes in oxygen and carbo dioxide modulations.

FIG. 7 illustrates the results of the sensor implanted and interrogated in vivo in swine tongue ischemia model. Three sensors were implanted at a consistent depth of 5 mm in the lateral margin of the tongue. The sublingual artery was occluded through applying a tourniquet, which was released, and the tongue was subjected to manipulations. A. The sensors have responded appropriately to manipulation of circulation, and reported the oxygenation as expected. The sensors have immediately detected the application of the tourniquet occluding the sublingual artery, then they detected the release of the tourniquet and the lower oxygenation resulted from damaging the sublingual artery. B. Tongue dissection post euthanasia demonstrated consistent implantation depth of sensors. C. Fluorescent imaging of the post-mortem swine tongue to illustrate the location of the sensors.

FIG. 8 illustrates the sensors of the disclosure implanted in different swine skin regions (right forelimb, highest starting oxygen tension; chest, the mid-level starting oxygen tension; left hind limb, the lowest starting oxygen tension) to monitor oxygenation during euthanasia.

FIG. 9 illustrates in vivo rat skin flap experiment. One day before surgery, three sensors were intradermally implanted at tip, middle and base of the impending flap on the dorsum of the eight rats. One day later, the outlined, caudally-based, full thickness flap was elevated. Readings from the sensors were obtained on days −1, 0, 3 and 7 post-surgery. A. Fluorescent imaging of the sensors in a rat on days 0 and 28 post-surgery, demonstrating that the sensors have not migrated from the original implantation locations. B. Analysis has demonstrated that the sensors were able to detect significant decrease in oxygenation in the tip of the flap in comparison to the base at all time points (*p<0.05).

FIG. 10 illustrates the sensor of the disclosure implanted in a rat myocutaneous flap model. Sensors were implanted intradermally in the impending flap site of eight rats. Superficial inferior epigastric artery (SIEA) myocutaneous flaps were surgically elevated. The SIEA flap was first outlined on the shaved skin of the right ventral abdomen by placing a 3×5 cm square template based on the location of the superficial inferior epigastric vessels. These vessels were carefully dissected to create a 3×5 cm island flap containing skin, subcutaneous fat, and panniculus carnosus muscle. Tissue oxygen tension (TOT) readings were obtained from implanted sensors both at baseline and during vascular clamping of the feeding blood vessels. A. Real-time analysis of the sensors implanted in the myocutaneous flaps has demonstrated that acute vascular clamping of the feeding blood vessels in the pedicle were immediately detected within 70 seconds. (*p<0.05). B. Schematic illustrating the flap design and the location of the implanted sensor.

FIG. 11 illustrates the preparation of the oxygen sensor composition of the disclosure.

FIG. 12 illustrates an optical fiber device of the disclosure.

### DETAILED DESCRIPTION OF THE INVENTION

Before the disclosed processes and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the term “contacting” includes the physical contact of at least one substance to another substance.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient.

In view of the present disclosure, the methods and compositions described herein can be configured by the person of ordinary skill in the art to meet the desired need. In general, the disclosed materials, methods, and apparati provide improvements in real-time, in vivo monitoring of oxygenation. The inventors found a very efficient measurement of oxygen tension and/or oxygen concentrations in various tissues of the subject using the oxygen sensor compositions of the disclosure.

For example, once the oxygen sensor is in place, oxygen tension and/or oxygen concentration can be measured non-invasively at the site of implantation. The sensors may be implanted in either the skin or subcutis via a single needle stick. The sensors allow for real-time monitoring of localized oxygen tension at the particular site where the sensors are implanted, and also allow for rapid detection of changes in oxygenation. In many clinical scenarios, such as surgical flaps, the maintenance of adequate skin oxygenation is crucial to the success of the surgery. Identification of hypoxia allows for timely corrective action to restore oxygenation and salvage a compromised flap. It is estimated that 6 to 25 percent of skin flaps require a secondary surgical intervention and around 10 percent of flaps fail. Non-invasive monitoring of changes in tissue oxygenation where the sensors are injected, thus, obviates the need for percutaneous and cabled monitoring. In addition, the oxygen sensor compositions of the disclosure may also be biodegradable.

Most optical oxygen sensors are based on the decrease in fluorescence or phosphorescence intensity of a fluorophore(s) (indicator(s)) when they are quenched by molecular oxygen in either gas phase or in dissolved form. Some sensors are based on the fluorescence or phosphorescence lifetime decrease upon exposure to oxygen. The relationship between the intensity or lifetime in the absence (I₀, τ₀) and presence (I, τ) of oxygen is described by the Stern-Volmer equations:

I₀/I=1+K_{SV}[O₂] (1)

τ₀/τ=1+kₛᵥ[O₂] (2)

where Kₛᵥ is the Stern-Volmer quenching coefficient having a specific value for each fluorophore/quencher system. [O₂] is the concentration of O₂, and when in gas phase, it is the partial pressure of oxygen (pO₂) and the oxygen solubility (concentration) in water (in ppm) while in aqueous phase. Because oxygen can quench the fluorescence of many fluorophores, the quenching of the fluorescence is used for the detection of oxygen.

A similar premise is used for the present disclosure. The fluorescence of the metalloporphyrin compounds of the present disclosure is also quenched through the binding of oxygen, and hence can be used for the detection of oxygen. The polymer modification of the metalloporphyrin enables the metalloporphyrin to become fluorescent. The spectral characteristics (e.g., excitable wavelengths and emittance wavelengths) of the oxygen-sensing compound are dependent on the type of porphyrin and/or transition metal and/or polymer used.

Thus, one aspect the present disclosure provides an oxygen-sensing compound including a metalloporphyrin encapsulated within a polymer particle. Such oxygen-sensing compounds enable the measurement of oxygen concentration in a non-invasive, real-time, and continuous manner.

The disclosure also provides an oxygen sensor composition including an oxygen-sensing compound embedded within a hydrogel carrier, wherein the oxygen-sensing compound includes a metalloporphyrin encapsulated within a polymer particle.

As provided above, the oxygen-sensing compounds incorporate metalloporphyrin compound that is capable of fluorescing and/or phosphorescing with an intensity and lifetime that correlates with the degree of oxygenation.

In certain embodiments, the metalloporphyrin may be a compound including a macrocyclic tetra pyrroles and their variations/modification that are able to incorporate a transition metal.

In some embodiments, the metalloporphyrin further comprises a transition metal. As used herein, the term “transition metal” refers to one of the 38 elements in groups 3 through 12 of the periodic table. Transition metals suitable for the compositions of the disclosure include, but are not limited to, candium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, etc. In some embodiments, the transition metal is palladium.

In certain embodiments, the metalloporphyrin comprises palladium tetraphenyl-tetrabenzoporphyrin (PdTPTBP).

As provided above, the metalloporphyrin is encapsulated within a polymer particle in the oxygen-sensing compounds of the disclosure. The polymer may comprise any polymer capable of encapsulating the metalloporphyrin thereby providing stability and retaining (or helping to impart) desirable optical properties. Suitable polymers may include, but are not limited to, polyethylethylene, poly(butadiene), poly(β-benzyl-L-aspartate), poly(lactic acid), poly(propylene oxide), poly(ε-caprolactam), oligo-methacrylate, polystyrene, polycaprolactone, polylactide, polyglycolide, poly(ethylene oxide)-polyethylethylene, poly(ethylene oxide)-poly(butadiene), poly(ethylene oxide)-poly(ε-caprolactone), poly(ethylene oxide)-poly(lactic acid), poly(vinyl chloride), poly(methyl methacrylate), poly(propylene) combinations thereof, and the like. In certain embodiments, the polymer is selected from the group poly(vinyl chloride), poly(methyl methacrylate), poly(propylene), polystyrene, and combinations thereof. In certain embodiments, the polymer comprises polystyrene.

The person of ordinary skill in the art will appreciate that a given polymer may have a variety of molecular weights and structures. Unless otherwise indicated, a “molecular weight” as used throughout is “weight-average” molecular weight, M_{W}. M_{W} may be calculated by using the equation: ΣMᵢ²nᵢ/ΣMᵢnᵢ, where nᵢ is the number of molecules of molecular weight Mᵢ. The M_{W} can be determined using any known technique, such as light scattering, small angle neutron scattering, X-ray scattering, or sedimentation velocity. The structures provided herein represent a weight average structure over the sample of the polymer. The person of ordinary skill in the art will be able to distinguish between different polymers, as having substantially different average molecular weights, or substantially different structures.

In certain embodiments, the polymer has a M_{W} of about 500 Da to about 20 kDa. For example, the polymer has a M_{W} of about 1 kDa to about 10 kDa; or about 1 kDa to about 5 kDa, or about 5 kDa to about 10 kDa, or about 3 kDa to about 7 kDa.

In certain embodiments, metalloporphyrin may be loaded into the polymer particle in the range of about to about 0.5 wt % to about 30 wt %, based on the total weight of the polymer. For example, metalloporphyrin may be loaded into the polymer particle in the range of about 1 wt % to about 20 wt %, or about 1 wt % to about 18 wt %, or about 1 wt % to about 15 wt %, or about 1 wt % to about 12 wt %, or about 1 wt % to about 10 wt %, or about 1 wt % to about 8 wt %, or about 1 wt % to about 5 wt %, or about 5 wt % to about 20 wt %, or about 5 wt % to about 18 wt %, or about 5 wt % to about 15 wt %, or about 5 wt % to about 12 wt %, or about 5 wt % to about 10 wt %, or about 5 wt % to about 8 wt %, or about 10 wt % to about 20 wt %, or about 10 wt % to about 15 wt %, or about 15 wt % to about 20 wt %.

The polymer particle may be of various sizes. For example, in some embodiments, the particle is a microscale particle. For example, the particle has a diameter of more than 100 μm. In other embodiments, the particle is a microscale particle. For example, the particle has a diameter in the range of about 0.1 μm to about 100 μm; e.g., in the range of about 0.1 μm to about 50 μm, or about 0.1 μm to about 20 μm, or about 0.1 μm to about 10 μm, or about 0.1 μm to about 5 μm, or about 1 μm to about 100 μm, or about 1 μm to about 50 μm, or about 1 μm to about 20 μm, or about 1 μm to about 10 μm, or about 1 μm to about 5 μm, or about 1 μm to about 3 μm, or about 2 μm to about 3 μm, or about 10 μm to about 100 μm, or about 10 μm to about 50 μm. In yet another embodiment, the particle is nanoscale particle. For example, the particle has a diameter in the range of about 1 nm to about 100 nm; e.g., in the range of about 1 nm to about 50 nm, or about 1 nm to about 20 nm, or about 1 nm to about 10 nm, or about 1 nm to about 5 nm, or about 1 nm to about 3 nm, or about 2 nm to about 3 nm, or about 10 nm to about 100 nm, or about 10 nm to about 50 nm, or about 50 nm to about 100 nm.

The oxygen-sensing compound of the disclosure may be prepared by any method known in the art. In a non-limiting example, the metalloporphyrin (e.g., PdTPTBP) may be combined with a polymer (e.g., polystyrene) carrier in chloroform is then cast as a thin film which is allowed to dry. The resulting solid is a powder containing the oxygen-sensing compound (e.g., metalloporphyrin encapsulated in the polymer particle).

As provided above, the oxygen sensor composition of the disclosure includes a hydrogel carrier. Any suitable hydrogel can be used to encapsulate the particle. For medicinal uses, the hydrogel is preferably biocompatible. Further, the hydrogels may be capable of reversible deformation. For example, the hydrogel may be implanted dry (e.g., in a smaller size/amount) and allowed to swell once implanted. Importantly, the hydrogels may maintain their original 3-dimensional shape and size making them easier to implant. Further, the particles are not covalently bound to the hydrogel carrier.

The hydrogel carrier may comprise a second polymer. Examples of the second polymer include, but are not limited to, poly(ethylene glycol) (PEG), poly(ethylene glycol) diacrylate (PEGDA), poly(hydroxethyl methacrylate) (PHEMA), silicone, poly(dimethylsiloxane) (PDMS), alginate, agarose, hyaluronic acid/hyaluronan, and their various formulations. In some embodiments, the second polymer comprises PEG diacrylate (PEGDA) (and various forms thereof). In certain embodiments, PEGDA is in its native nanoporous form. In certain embodiments, PEGDA is in its microporous form.

In certain embodiments, the second polymer has a M_{W} of about 500 Da to about 20 kDa. For example, the second polymer has a M_{W} of about 1 kDa to about 10 kDa; or about 1 kDa to about 5 kDa, or about 5 kDa to about 20 kDa, or about 5 kDa to about 10 kDa, or about 3 kDa to about 10 kDa, or about 3 kDa to about 7 kDa.

The hydrogel carrier may a protein or peptide hydrogel, such as collagen, gelatin, fibrin, elastin, bovine serum albumin (BSA), human serum albumin (HSA), etc.

In some embodiments, the number of particles within the hydrogel carrier may be varied to account for numerous factors, such as amount of fluorescence or phosphorescence needed for detection (e.g., deep tissue), amount of O₂ present in the sample, etc. In certain embodiments, the particle may be loaded into the hydrogel carrier in the range of about to about 1 wt % to about 50 wt %, based on the total weight of the hydrogel. For example, the particle may be loaded into the hydrogel carrier in the range of about to about 1 wt % to about 40 wt %, or about 1 wt % to about 30 wt %, or about 1 wt % to about 25 wt %, or about 10 wt % to about 50 wt %, or about 10 wt % to about 40 wt %, or about 10 wt % to about 25 wt %, or about 10 wt % to about 20 wt %, or about 25 wt % to about 50 wt %, or about 25 wt % to about 40 wt %, or about 25 wt % to about 30 wt %, or about 15 wt % to about 35 wt %, or about 20 wt % to about 30 wt %, or about 10 wt % to about 20 wt %, or about 10 wt % to about 15 wt %, or about 15 wt % to about 20 wt %.

The hydrogel of the disclosure may be prepared by any method known in the art. In a non-limiting example, hydrogels formed at room temperature may be made into nanoporous hydrogels. In another non-limiting example, hydrogels formed at below zero temperatures (e.g., −20° C.) may be made into microporous cryogels. The cold polymerization creates competition between polymerization and ice crystal formation which leads to its microporous structure, which increases the toughness of the hydrogel. This toughness allows for the hydrogel to be injected within a tissue of a subject.

In another non-limiting example, hydrogels formed in emulsions may be made into nanoporous microspheres. For example, the nanoporous microspheres may have a diameter in the range of about 10 μm to about 100 μm; e.g, in the range of or about 50 μm to about 100 μm, or about 10 μm to about 50 μm, or about 30 μm to about 80 μm. In this formulation, the polymer particles may also be incorporated into microspheres through the use of an oil/water emulsion polymerization. This may also be similarly achieved via spray polymerization as well as microfluidics. In certain embodiments, the nanoporous microspheres may be further incorporated into other materials, such as suture materials as propyl propelene and poly urethane, and materials used for surgical devices, such as intravascular lines and indwelling devices.

The structure of the hydrogel may be used to control the degradation time of the oxygen-sensing compounds. For example, in certain embodiments, the second polymer monomers may incorporate variable numbers of bonds cleavable under physiological conditions, such as ester bonds (such aslactic acid, glycolic acid, or their combinations to the polymer) and disulfide bonds. For example, in certain embodiments, the second polymer may incorporate peptide sequences into the polymer backbone that can be selectively cleaved at different rates by various proteases including, but not limited to, matrix metalloproteinases, plasmins, and cathepsins. Each of these modes of degradation can be done in conjunction with any of the polymerization schemes.

In certain embodiments, the sensor composition of the disclosure may further include one or more different types of sensing compounds such as, but not limited to those specific for pH, CO₂, O₂, potassium, sodium, lactate, creatinine, glucose, urea, etc.

It will further be appreciated by persons skilled in the art that the oxygen sensor compositions of the disclosure may also comprise a suitable pharmaceutical excipient diluent or carrier selected with regard to the intended route of administration and standard pharmaceutical practice (for example, see Remington: The Science and Practice of Pharmacy, 19^{th} edition, 1995, ed. Alfonso Gennaro, Mack Publishing Company, Pennsylvania, USA).

For example, for application topically, e.g. to the skin or a wound site, the compositions of the present disclosure may be formulated as a suitable ointment containing the active compound suspended or dissolved in, for example, a mixture with one or more of the following: mineral oil, liquid petrolatum, white petrolatum, propylene glycol, polyoxyethylene polyoxpropylene compound, emulisifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, e-lauryl sulphate, an alcohol (e.g. ethanol, cetearyl alcohol, 2-octyldodecanol, benzyl alcohol) and water. In certain embodiments, the formulation (e.g. lotion, solution, cream, gel or ointment) is water-based.

The oxygen sensor compositions of the disclosure may also be formulated for parenteral administration (for example, for administration intravenously, intra-arterially, intraperitoneally, intrathecally, intraventricularly, intrasternally, intraeranially, intra-muscularly or subcutaneously (including via an array of fine needles or using needle-free Powderject® technology), or by infusion techniques). In some embodiments, the oxygen sensor compositions of the disclosure may be in form of a cryogel as described herein, where the oxygen sensor composition is suspended in a cryogel and injected into the subject. In other embodiments, the oxygen sensor compositions of the disclosure may take the form of a sterile aqueous solution which may contain other substances, for example, an enough salts or glucose to make the solution isotonic with blood. The aqueous solutions should be suitable buffered (preferably to a pH of from 3 to 9), if necessary. The preparation of suitable parenteral formulations under sterile conditions is readily accomplished by standard pharmaceutical techniques well-known to those skilled in the art.

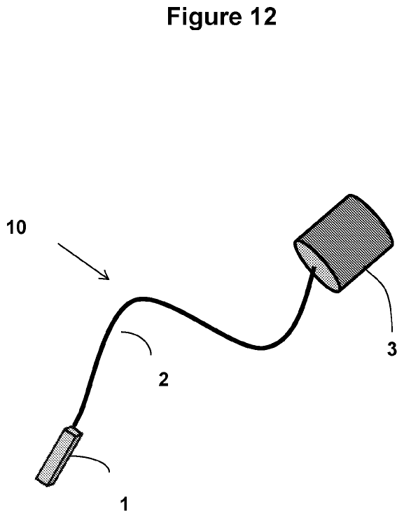

Another aspect of the disclosure provides an optical fiber device. The optical fiber devices of the disclosure may be placed in locations that would not normally be directly optically accessible, such as in, but not limited to, deep body organs like the heart, lungs, liver, and kidneys. Referring to FIG. 12, the optical fiber device 10 comprises, consists of, or consists essentially of an optic probe 1, where the surface of the probe 1 is coated with the sensor composition of the disclosure. Probe 1 is optically coupled with to the tip of an optical fiber 2. The end of the optical fiber 2 is coupled to a remote detector 3. In certain embodiments, a remote detector 3 may provide the excitation light and/or collect the emitted light. The optical fiber 2 transmits the excitation light from a distant source to the probe 1 and transmits the emitted light from the probe 1 to a remote detector 3.

The optical fiber 2 may be an optical fiber bundle. The bundle may include optical fibers covered by protective layers. Generally, the optical fiber bundle may include several thousands to several hundred and thousand optical fibers which are several μm in diameter, respectively. Both ends of the optical fiber 2 may be equipped with reflection-proofed glass plates to prevent reflection at both ends.

It is also within the scope of present disclosure that the oxygen sensor composition of the disclosure described herein may also be incorporated into/onto other medical devices where the real-time monitoring of oxygen concentration would be desirable. Such device may include, but are not limited to, endotracheal tubes (to monitor real-time lung oxygenation), arterial blood lines (to monitor real-time arterial blood gases), intravenous lines, central venous catheters, contact lenses, urinary catheters, surgical sutures, pacemakers, all forms of implantable devices, and orthopedic fixation devices. The oxygen sensor composition of the disclosure may also be incorporated into wound dressings and wound vacuum dressings thereby allowing for the real-time measurement of oxygenation in the wound. The oxygen-sensing compounds according to the present disclosure may also be used in numerous neurological applications, including, but not limited to, the continuous oxygen monitoring in cases of subarachnoid hemorrhages and intracranial hemorrhages. Further, the oxygen-sensing molecules (in, for instance, a hydrogel) can be implanted to predict/monitor peripheral vasospastic disease such as Raynaud's disease, acrocuanosis, livedo reticularis, and the like.

One aspect of the disclosure provides methods for monitoring oxygenation in a subject. In certain embodiments, such methods include: (i) administering to a subject a therapeutically effective amount of an oxygen sensor composition of the disclosure; (ii) activating an excitation light source to excite the oxygen-sensing compound; (iii) measuring the fluorescence or phosphorescence (e.g., the emitted light) from the oxygen-sensing compound; and (iv) calculating the concentration of oxygen from the measurement. In certain embodiments, such methods include: (i) coating a tip of an optic probe with an oxygen sensor composition of the disclosure; (ii) inserting the optic probe into the desired deep tissue of the subject; (iii) activating an excitation light source to excite the oxygen-sensing compound; (iv) measuring the fluorescence or phosphorescence (e.g., the emitted light) from the oxygen-sensing compound; and (v) calculating the concentration of oxygen from the measurement.

The methods of the disclosure allow for real-time and/or continuous measurement. The methods of the disclosure also allow for in vivo measurement in the tissue.

In certain embodiments, the measurement may be in the deep tissue.

In some embodiments, the deep tissue is selected from the group consisting of heart, lungs, liver, kidneys and combinations thereof.

The methods of the disclosure may also be used clinically in many diseases requiring monitoring of oxygen concentration in a certain organ or tissue. For example, in clinical scenarios like monitoring flap viability in the postoperative period, the oxygen sensor composition of the disclosure will immediately detect any vascular compromise through detecting decreases in oxygenation and thus can warrant an intervention to salvage the flap. Accordingly, in certain embodiments, the tissue is a surgical flap, replanted tissue, or transplanted organ of a subject.

Yet another application is in peripheral artery disease, where a plaque forms that can block arteries and require implantation of a vascular bypass. In such embodiments, the oxygen sensor compositions according to the disclosure are used to monitor oxygen concentration of affected tissues. Hence, another embodiment of disclosure provides methods wherein the tissue is in and around the peripheral arteries.

The oxygen sensor compositions of the disclosure may be included in numerous other methods that require the real-time monitoring of oxygen concentration. Such methods include, but are not limited to, free tissue transfer, organ transplants, hand and digital replant procedures, diabetic ulcers, pulmonary diseases (e.g., COPD), the monitoring of tumor response to treatment (and the prediction of prognosis and/or drug efficacy), skin grafts, vascular grafts and vascular bypass surgeries, dialysis shunts, the monitoring of decubitus ulcers, the monitoring of cyanosis in newborns, the monitoring if tongue necrosis during endotracheal intubation, and the like.

Further, it is within the scope of the present disclosure that the oxygen sensor compositions described herein may also be incorporated into non-in vivo/biological uses, such as for the monitoring of cell/bacterial cultures, monitor the viability of stem cells, incorporating into tattoo ink, and the like. The oxygen sensor compositions of the disclosure also have applicability in the monitoring of oxygen concentrations in open/closed systems, such as water flow, fermentation, bioreactors and the like.

Another aspect of the disclosure provides systems for monitoring oxygenation. Such systems include (i) an oxygen sensor composition of the disclosure; (ii) an excitation light source; and (iii) an instrument for measuring and reporting fluorescence or phosphorescence from the activated oxygen-sensing compound.

The systems of the disclosure allow for real-time and/or continuous measurement. The systems of the disclosure also allow for in vivo measurement in the tissue.

In certain embodiments, the systems may be adapted for measurement in the deep tissue. For example, the system of the disclosure may include the optic probe as described herein.

### EXAMPLES

The composition and methods of the disclosure are illustrated further by the following examples, which are not to be construed as limiting the disclosure in scope or spirit to the specific procedures and compounds described in them.

The compositions disclosed herein can be made using procedures familiar to the person of ordinary skill in the art and as described herein. One of skill in the art can adapt the reaction sequences of the example below to fit the desired target molecule. Of course, in certain situations one of skill in the art will use different reagents to affect one or more of the individual steps or to use protected versions of certain of the substituents. Additionally, one skilled in the art would recognize that compositions of the disclosure can be synthesized using different routes altogether.

### Example 1: Synthesis of PEGDA

Poly(ethylene glycol) diacrylate (PEGDA) was synthesized by reacting 24 g poly(ethylene glycol) (PEG), 6000 MW (4 mmol, Fluka) with 1.27 mL acryloyl chloride (16 mmol, Sigma) and 1.12 mL triethyl amine (8 mmol, Sigma) overnight in 40 mL of dichloromethane (DCM) under argon atmosphere. The reaction was diluted with an additional 20 mL of DCM then washed with 2M K₂CO₃. The organic layer was collected and dried over MgSO₄. The PEGDA could then be precipitated in diethyl ether, filtered and dried. NMR was used to evaluate the degree of acrylation.

### Example 2: Synthesis of Polystyrene-Encapsulated Porphyrin Sensors

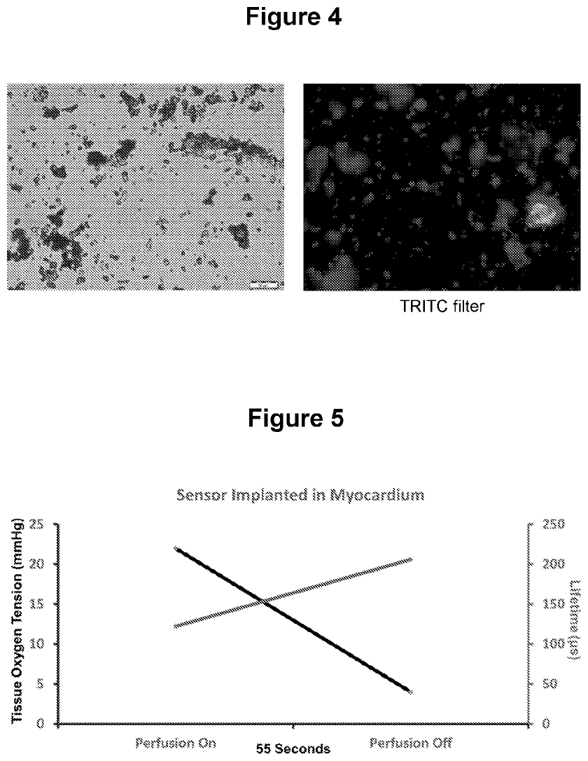

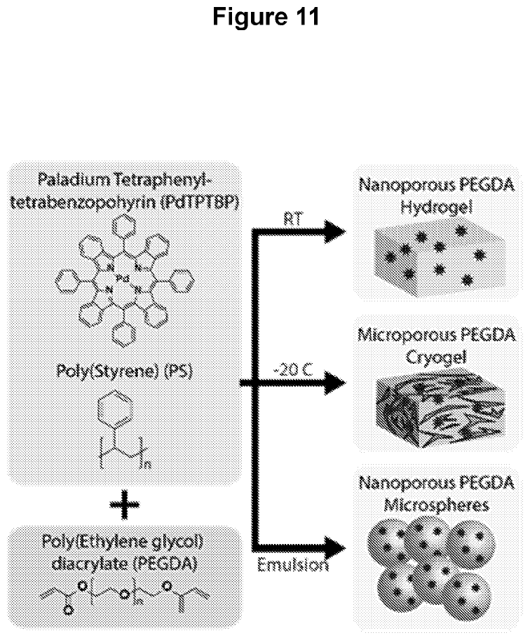

As generally illustrated in FIG. 11, Pd(II) tetraphenyltetrabenzoporphyrin (PdTPTBP, Adipogen), 1.8 mg, was mixed with 30 mg polystyrene (Mg=2960 Da, Polymer Labs) and dissolved in 450 μL of chloroform. The solution was cast as a thin film against a clean glass slide and allowed to dry overnight. The particles obtained, which are shown in FIG. 4, had average mean particle size of 2.5 μm. The film was removed and titurated then mixed with 100 mg PEGDA and 1 mL of Milli-Q H₂O. The solution was thoroughly mixed before adding 0.25% (w/v) ammonium persulfate and 0.5% (w/v) TEMED.

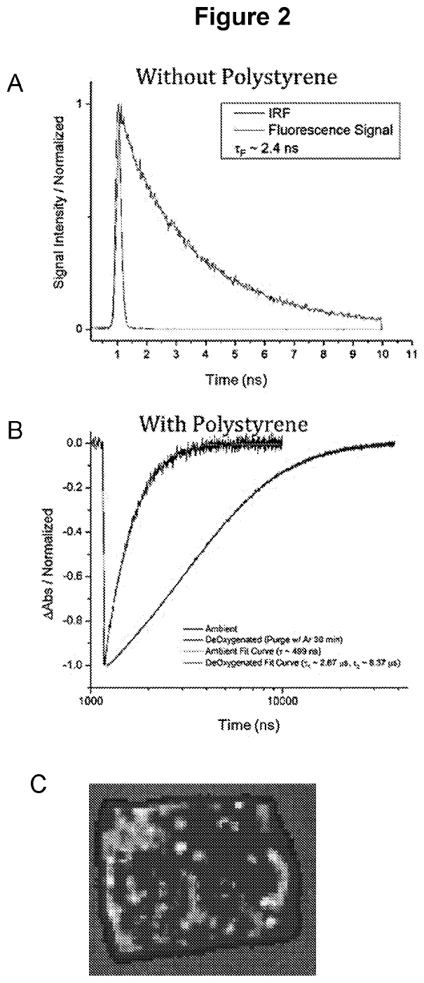

For nanoporous gels, the solution was mixed and pipette between two clean glass slides sandwiching a 1 mm thick Teflon mold and allowed to polymerize at room temperature for 30 minutes. The porphyrin-embedded hydrogels were then rinsed several times in milli-Q H₂O and cut into the necessary dimensions for subsequent experiments. Hydrogels were stored at 4° C. in sterile water until use. Homogenous distribution of the metalloporphyrin was confirmed through fluorescent imaging via IVIS kinetic imaging system (FIG. 2C).

For microporous gels, the solution was pipette into a similar mold that had been pre-cooled at −20° C. and allowed to polymerize overnight at −20° C. The porphyrin-embedded hydrogels were then rinsed several times in milli-Q H₂O and cut into the necessary dimensions for subsequent experiments. Hydrogels were stored at 4° C. in sterile water until use.

Spectroscopic Analyses.

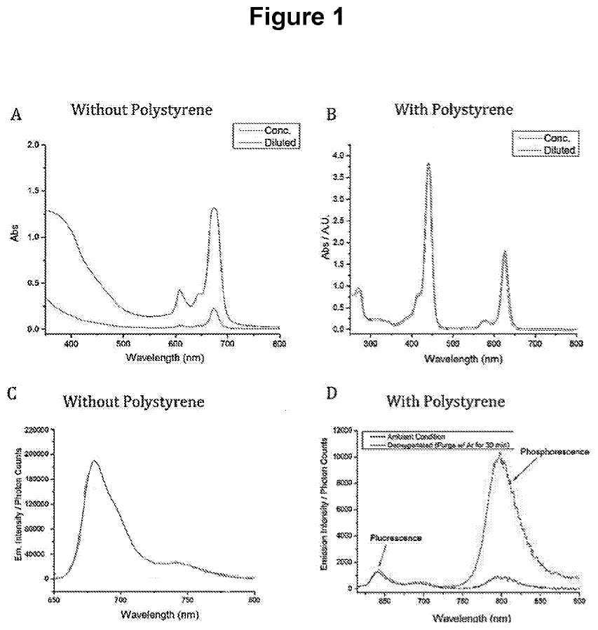

Electronic absorption spectra were recorded on a Shimadzu UV-1700 spectrophotometer; sample was dissolved in THF solvent in a 10 mm quartz optical cell. Steady state emission spectra were recorded on a FLS920 spectrometer that utilized a xenon lamp (Xe900) as excitation light source and an extended red sensitive PMT (Hamamatsu R2658P side window photomultiplier, spectral range: 200-1010 nm) for detection. Emission spectra were corrected using calibration curve supplied with the instrument. Porphyrin compound was dissolved in THF solvent in 10 mm quartz optical cell, and investigated at ambient temperature in both deoxygenated and oxygenated condition. Deoxygenation was achieved through purging with dry argon gas over ˜30 min. The optical density at excitation wavelengths is ˜0.1 (FIG. 1A-D).

Nanosecond Spectroscopic Analysis.

The photoluminescence lifetime was acquired utilizing an Edinburgh Instruments LP920 Laser Flash Photolysis Spectrometer and Edinburgh L900 Software. Pump pulses were generated from a Q-switched Nd:YAG laser (Quantel, Brilliant) and a dual-crystal OPO (OPOTEK, Vibrant LDII). The temporal width of the pump pulses was ˜5 ns; the energy of the pulses exiting the OPO was controlled using neutral density filters. A Xe flash-lamp was used as a white light probe source. Both the LP920 and Opotek OPO are computer interfaced and controlled by the L900 software. Kinetics reported derive from data acquired over ˜20-50 scans. Samples were prepared in 1 mm quartz cells and purged with dry argon gas or air prior to excitation. Excited-state lifetimes were calculated via simple exponential fitting using Origin software. (FIG. 2A-B).

Combining the oxygen-sensitive porphyrins with polystyrene led to the formation of microparticles with spectroscopic properties which are different from the original oxygen-sensing porphyrin. These new resulting spectroscopic propertied allowed for enhancing the signal and obtained from the resulting compound, and allowed for a favorable shift in the absorption and emission wavelength, as well as increasing the lifetime of fluorescent decay (phosphorescence) which increases the usability for deeper implantation and usability of the sensors.

The resulting polystyrene-encapsulated porphyrins demonstrate more steady state fluorescence intensity as opposed to the porphyrin alone (FIG. 1A-B). The polystyrene-encapsulated porphyrins demonstrate shift increase in the emission peak which improves collection of the emitted signal through deeper implantation depth (FIG. 1C-D). The polystyrene-encapsulated porphyrins also demonstrate increase in the lifetime of fluorescence decay from nanoseconds to microseconds which increases the range of detecting changes in oxygenation and hence increase the possibility of using the microparticles for the use in physiologic and pathologic conditions. (FIG. 2A-2B).

### Example 3

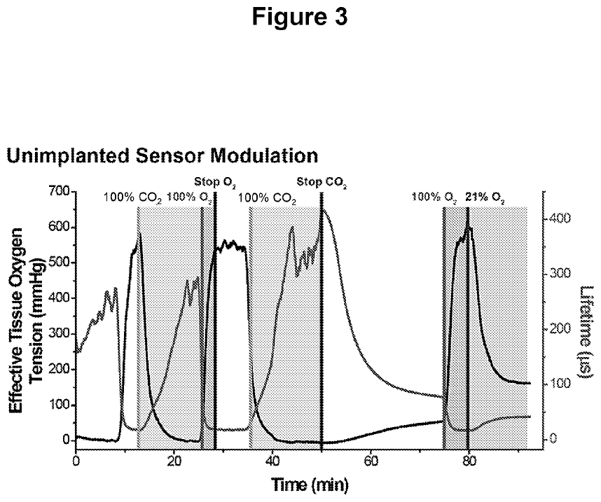

To determine the responsiveness and sensitivity, the non-implanted polystyrene-encapsulated porphyrin sensors were placed in a closed system that was sequentially purged with 100% CO₂ followed by 100% O₂. This process was then repeated and allowed to equilibrate to ambient oxygen. Here, the changes in ambient oxygen directly correlated to changes in the porphyrin's phosphorescence lifetime to generate an effective oxygen concentration (FIG. 3).

In addition, these same sensors were then placed in normal saline solution for about 9 months. The signal and modulation from these molecules did not show unexpected variation or decreased signal.

### Example 4: Synthesis of Synthesis of Biodegradable Sensor

Porphyrin-PEG-PQ was created by encapsulating the porphyrin/polystyrene microparticles in a PEGDA hydrogel that incorporates a matrix metalloproteinase (MMP)-sensitive peptide, GGGPQGIWGQGK (SEQ ID NO: 1; abbreviated PQ). Here, mono-acrylate-poly(ethylene glycol)-succinimidyl valerate was coupled to the amine-terminus of the peptide as well as to the C-terminus via the terminal lysine residue. The porphyrin/polystyrene microparticles (average mean particle size of 2.5 μm) were added to the generated PEG-PQ-PEG diacrylate macromer, and the mixture was then crosslinked in the presence of a chemical initiator (ammonium persulfate/tetramethylethylenediamine). This created a highly crosslinked hydrogel network that resists protein adsorption and permits the selective degradation of the hydrogel carrier.

It is expected that the hydrogel degrades within one month post implantation. The degraded poly(ethylene glycol) is cleared, leaving the <0.2 μl of porphyrin/polystyrene particles behind. This remaining particle mixture equates to about 10 μg of porphyrin (assuming a 5 μL hydrogel implant), which is about 1.2 μg of palladium coordinated within the porphyrin. This amount of palladium is much below the accepted levels for parenteral palladium administration (i.e., parenteral administration not exceeding 10 μg/day and oral administration not exceeding 100 μg/day). Therefore, the compositions of the disclosure permit pre-procedure baseline measurements, real-time tissue oxygen measurements during procedures, as well as post-procedural monitoring for up to several weeks. The biodegradable formulation is appropriate for short-term clinical applications, such as skin flaps, where oxygen tension monitoring is helpful during the first week post-procedure, after which neovascularization makes further monitoring unnecessary.

### Example 5: Ex Vivo Testing in Swine Heart

To determine the responsiveness and sensitivity of the sensors ex vivo, a fresh swine heart transplant model was used. Briefly, a fresh swine heart was harvested to be prepared for transplant in another pig. The sensors were implanted via a 19G needle in the heart that was being perfused on an ex vivo heart perfusion pump that keeps the heart pumping. The sensor was implanted in the myocardium of the left ventricle at a depth of ˜5 mm. The real-time readings fluorescent lifetime decay was obtained by a real-time fluorescent reader. Readings were obtained while the heart was being perfused and after perfusion was discontinued.

As shown in FIG. 5, the sensors appropriately and consistently responded and could detect the acute decrease in perfusion which has demonstrated as acute decrease in oxygen tension. Ex. 2 micorporous sensors were implanted in the left myocardium of and ex-vivo swine heart being perfused by a heart pump perfusion device, which supplies oxygen to the heart. Oxygen readings through fluorescent lifetime decay were obtained while the perfusion was on, and after stopping the perfusion. The sensors were able to detect high oxygen tension while the perfusion is on, and they were able to detect an acute decrease in oxygenation within 55 seconds of stopping the perfusion.

### Example 6: Ex Vivo Testing in Swine Skin

To determine the responsiveness and sensitivity of the sensors, an ex vivo swine skin model was utilized. Briefly, a rectangular 10×10 cm rectangular piece of fresh swine back skin was obtained. The sensors of Example 2 (microporous cryogel) were implanted via a 19G-size needle intradermally (4 mm deep) and subcutaneously (6 mm deep) in the swine skin. Absolute fluorescence was imaged via the IVIS kinetic imaging system, and the fluorescent lifetime decay reading was done via a real-time fluorescence reader. The swine skin specimen, containing the implanted sensors was placed in a closed system that was sequentially purged with 100% O₂ followed by 100% CO₂.

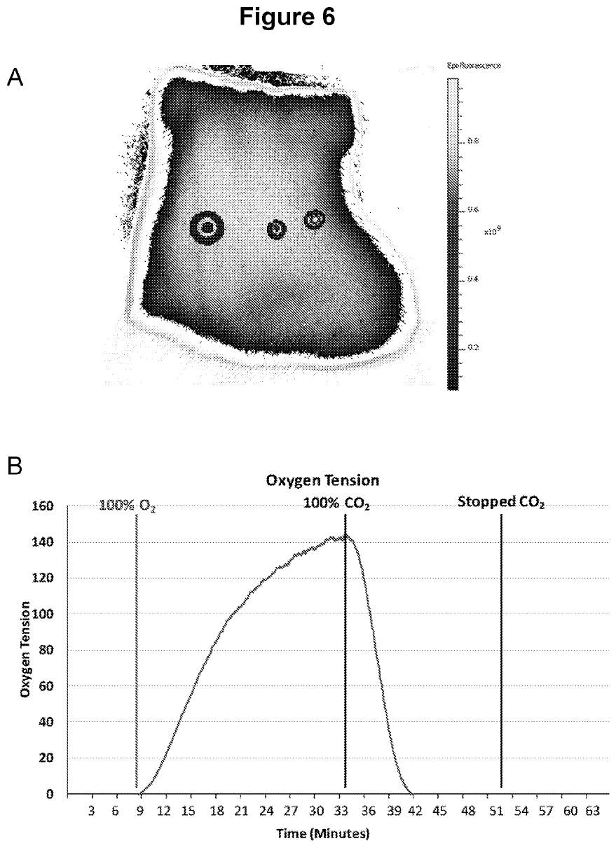

As provided in FIG. 6A, absolute fluorescence was able to detect strong signal from the sensors. Real-time fluorescent lifetime decay was observed, and the sensors have modulated appropriately as expected in response to changes in O₂ and CO₂ (FIG. 6B). The sensors were implanted in different depth (3 mm, 5 mm, and 7 mm deep), fluorescent imaging was able to detect signal from the sensors. Later, the swine skin was placed in saline solution, and 100% O₂ was bubbled for 24 minutes, followed by CO₂ bubbling for 9 minutes. Oxygen tension readings using real-time fluorescence lifetime decay from the deep subcutaneous sensor. The oxygen sensor has appropriately responded to changes in oxygen and carbo dioxide modulations.

### Example 7: In Vivo Testing in Swine Tongue

To determine the responsiveness and sensitivity of the sensors in vivo, three sensors of Example 2 (microporous cryogel) were implanted in vivo in swine tongue (n=3). Briefly, a swine acute tongue necrosis model was utilized using a yorkshire pig. While anesthesized, the sensors were implanted in the tongue of the pig a depth of 5 mm using a 19G-size needle, the sensors were implanted in the middle portion of the tip, center, and base of the tongue, respectively. A variety of manipulations, including vascular occlusion through a tourniquet, release of the tourniquet and tongue massage, was performed on the tounge. This experiment measured fluorescence lifetime, which is a measure of the time a fluorophore spends in the excited state before returning to the ground state by emitting a photon. The emission is then collected by the optical analysis device and values were converted into tissue oxygen tension in mmHg.

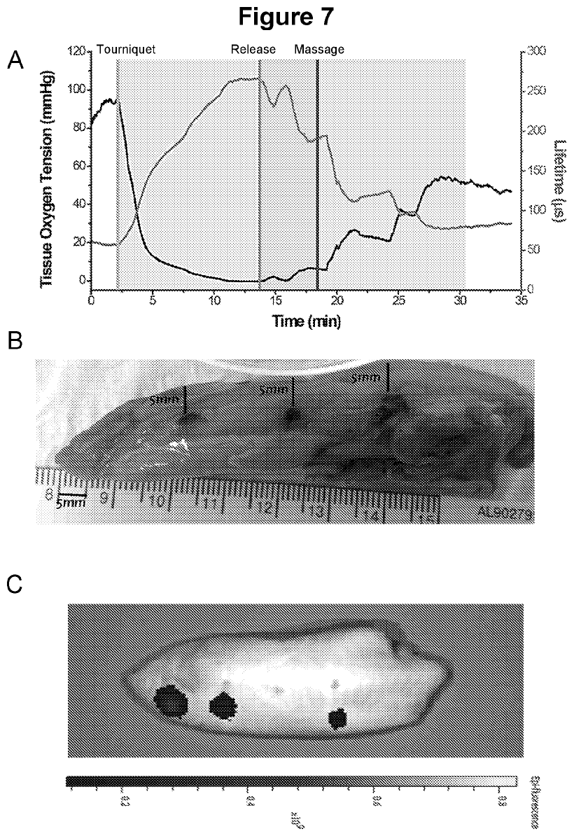

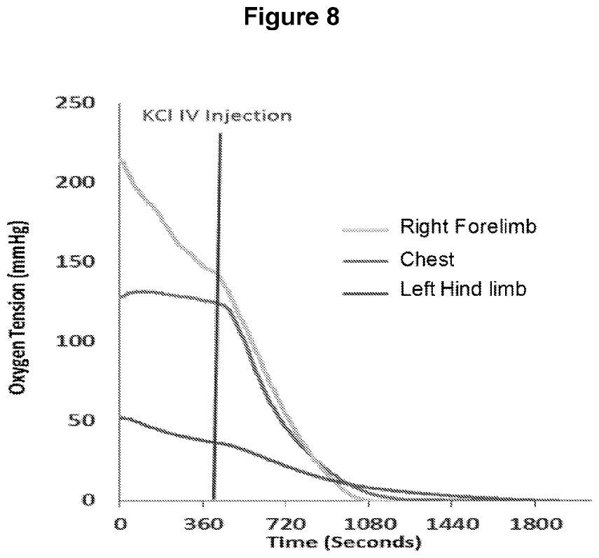

The sensors have appropriately responded to applying the tourniquets through reporting acute significantly decreased levels of oxygen. Three sensors were implanted at a consistent depth of 5 mm in the lateral margin of the tongue. The sublingual artery was occluded through applying a tourniquet, which was released, and the tongue was subjected to manipulations. The sensors have responded appropriately to manipulation of circulation, and reported the oxygenation as expected. The sensors have immediately detected the application of the tourniquet occluding the sublingual artery, then they detected the release of the tourniquet and the lower oxygenation resulted from damaging the sublingual artery. After releasing the tourniquets, the oxygen levels started rising again, as reported by the sensors. As provided in FIG. 7A, the sensors were able to detect acute vascular compromise of the sublingual artery with sensitivity and accuracy suitable for in vivo use. At the end of the experiment, following euthanasia, the tongue was harvested, fluorescent imaging through the IVIS imaging system demonstrated fluorescent signals from the sensors. As provided in FIG. 7B, dissection to detect the implantation depth of the 3 sensors was done and confirmed to be 5 mm deep for all 3 sensors. Fluorescent imaging of the post-mortem swine tongue to illustrate the location of the implanted sensors.

### Example 8: In Vivo Swine Euthanasia Model

While anesthetized, the sensors of Example 2 (microporous cryogel) were implanted in a Yorkshire swine in normal epidermis of the chest, right forelimb and left hindlimb of the pig. Baseline oxygen readings were obtained using real-time fluorescent lifetime decay measurements. While obtaining readings, euthanasia of the pig was performed through injecting intravenous euthanasia agent (KCl).

As provided in FIG. 8, the sensors have successfully detected and measured the change and decrease in oxygenation as the pig is losing the vital functions. Three sensors were implanted in the chest, right forelimb and the left hind limb. Real-time monitoring using fluorescent lifetime decay was started and continued as the pig received the euthanizing KCl agent. Decreases in oxygen tension were detected within one minute of KCl injection, we continued the monitoring process until the tissue oxygen tension reached a plateau low levels, within 18 minutes post-euthanasia. Physiologically, the areas closer to the heart receive better perfusion, and these areas will be more representing of the heart function. Thus, the chest and the right forelimb demonstrated sharp decrease in oxygen as the pig is losing his vital functions, because the blood flow is more rapidly affected in these regions, compared to the left hindlimb which is more distant from the heart.

### Example 9: Rat McFarlane Flap Model

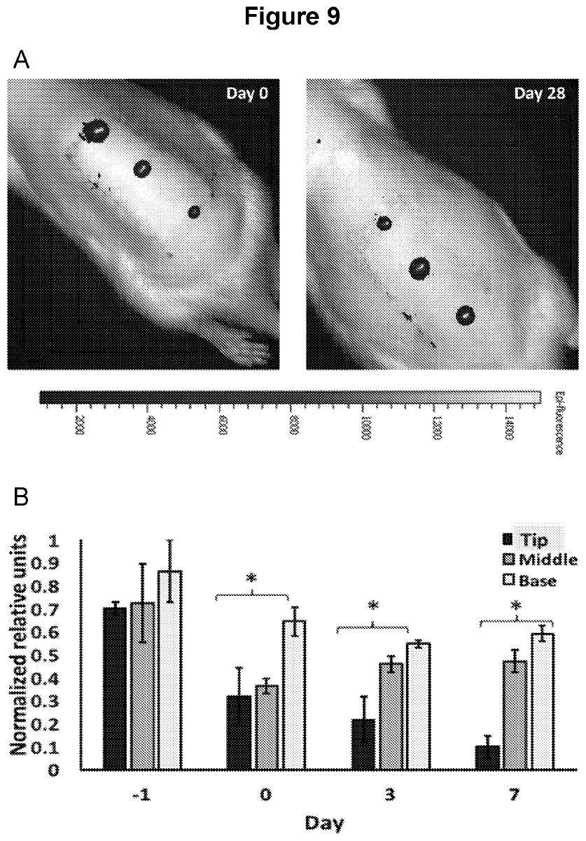

To mimic a clinical scenario in a rodent model, the sensors were implanted in a rat model of random flap. A previously validated and published McFarlane rodent random flap model was used (Briggs, P. C., 1987. THE McFARLANE FLAP. Plastic and Reconstructive Surgery 80, 472.). Briefly, male Sprague-Dawley rats had the skin flap site outlined and three sensors were intradermally implanted at tip, middle and base of the impending flap as provided in FIG. 9A. One day later, the outlined, caudally-based, full thickness flap was elevated on dorsum of rats. Absolute fluorescence readings from sensors of Example 2 (nanoporous) were obtained on days 0, 3, and 7 postoperatively using IVIS imaging system.

The sensors were able to predict flap necrosis on day 0 immediately after elevation of the flap as provided in FIG. 9B. The tip of the flap in this model experiences a very sharp decrease in perfusion and oxygenation because it is distant from the pedicle which is the only source for blood after elevating the flap. Readings in this experiment were obtained using absolute fluorescence imaging. The sensors were able to detect acute decrease in oxygenation in the tip of the flap which is designed in this model to be less vascular, compared to the base of the flap, which is more vascular. Other standard of care methods require lengthy time to detect decrease in perfusion. This is superior to current standard of care monitoring methods. In addition, fluorescence imaging of the sensors using an IVIS Kinetic device confirmed that sensors have not migrated for 28 days of the experiment duration. Readings were performed for 7 days post-operatively, because this is the critical clinical period to monitor flap viability. The implanted sensors were left in the model for 28 days to investigate whether the implants could migrate from the original implantation location in the skin. Findings confirmed that after 28 days, the sensors remained in the original location of implantation.

### Example 10: Rat Myocuteneous Flap Model

To assess how fast can the sensor respond to acute changes in oxygenation, a sensor was implanted in a rat myocutaneous flap model. Briefly, the sensors were implanted intradermally in the impending flap site. Superficial inferior epigastric artery (SIEA) myocutaneous flaps were surgically elevated. The SI EA flap was first outlined on the shaved skin of the right ventral abdomen by placing a 3×5 cm square template based on the location of the superficial inferior epigastric vessels. These vessels were carefully dissected to create a 3×5 cm island flap containing skin, subcutaneous fat, and panniculus carnosus muscle. Real-time fluorescent decay readings were obtained from implanted sensors of Example 2 (microprous cryogel), both at baseline and during vascular clamping of the feeding blood vessels.

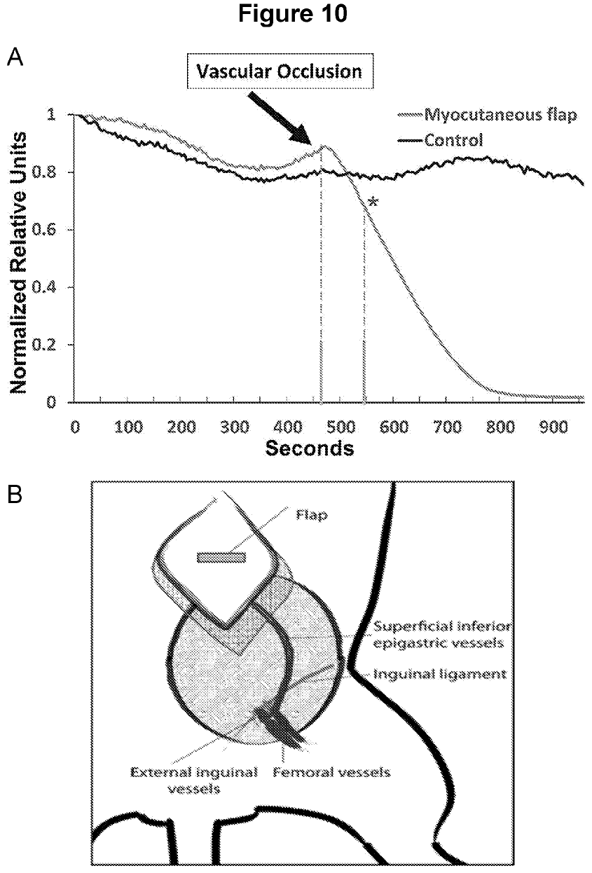

Clinical observation of the flaps did not show any significant change in color and temperature of the flaps during or immediately after clamping of the feeding blood vessels. As shown in FIG. 10A, real-time analysis of the sensors implanted in the myocutaneous flaps has demonstrated that acute vascular clamping of the feeding blood vessels in the pedicle were immediately detected within 70 seconds (*p<0.05). Real time readings were obtained using fluorescence lifetime decay.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

We claim:

1. An oxygen sensor composition comprising an oxygen-sensing compound embedded within a hydrogel carrier, wherein the oxygen-sensing compound comprises a metalloporphyrin encapsulated within a polymer particle.

2. The sensor composition of claim 1, wherein the metalloporphyrin comprises a transition metal.

3. The sensor composition of claim 2, wherein the transition metal is selected from the group consisting of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, and gold.

4. The sensor composition of claim 3, wherein the transition metal is palladium.

5. The sensor composition of any of claims 1-4, wherein the polymer is selected from the group poly(vinyl chloride), poly(methyl methacrylate), poly(propylene), polystyrene and combinations thereof.

6. The sensor composition of any of claims 1-4, wherein the polymer comprises polystyrene.

7. The sensor composition of any of claims 1-6, wherein the polymer has a Mw of about 500 Da to about 20 kDa; or about 1 kDa to about 10 kDa; or about 1 to about 5 kDa.

8. The sensor composition of claim 1, wherein the metalloporphyrin is palladium tetraphenyl-tetrabenzoporphyrin (PdTPTBP).

9. The sensor composition of any of claims 1-8, wherein the metalloporphyrin is loaded into the polymer particle in the range of about to about 1 wt % to 20 wt %, based on the total weight of the polymer.

10. The sensor composition of any of claims 1-9, wherein the particle has a diameter in the range of about 0.1 μm to about 100 μm.

11. The sensor composition of any of claims 1-9, wherein the particle has a diameter in the range of about 1 nm to about 100 nm.

12. The sensor composition of any of claims 1-9, wherein the hydrogel carrier comprises a second polymer selected from the group consisting of poly(ethylene glycol) (PEG), poly(ethylene glycol) diacrylate (PEGDA), poly(hydroxethyl methacrylate) (PHEMA), silicone, poly(dimethylsiloxane) (PDMS), alginate, agarose, hyaluronic acid/hyaluronan, and combinations and/or variations thereof.

13. The sensor composition of claim 12, wherein the second polymer is poly(ethylene glycol) diacrylate (PEGDA) or a variation thereof.

14. The sensor composition of claim 12 or 13, wherein the second polymer has a Mw of about 500 Da to about 20 kDa; or about 1 kDa to about 10 kDa; or about 4 to about 8 kDa.

15. The sensor composition of any of claims 1-14, wherein the particle is loaded into the hydrogel support in the range of about to about 1 wt % to 20 wt %, based on the total weight of the hydrogel support.

16. An optical fiber device for the detection of oxygen in a deep body organ of a subject comprising: (i) an optic probe that is coated with a sensor composition of any of claims 1-15; (ii) an optical fiber in electrical communication with the probe; and (iii) a remote detector electrical communication with the optical fiber.

17. A method for monitoring oxygenation in a subject, the method comprising:
(i) administering to a subject a therapeutically effective amount of a sensor composition of any of claims 1-15;
(ii) activating an excitation light source to excite the oxygen-sensing compound;
(iii) measuring the fluorescence or phosphorescence from the oxygen-sensing compound; and
(iv) calculating the concentration of oxygen from the measurement.

18. The method of claim 17, wherein the monitoring is in real-time, in vivo measurement in the tissue.

19. The method of claim 17 or 18, wherein the tissue is a deep tissue.

20. A method for monitoring oxygenation in a subject, the method comprising:
(i) coating a tip of an optic probe with a sensor composition of any of claims 1-15;
(ii) inserting the optic probe into tissue of the subject;
(iii) activating an excitation light source to excite the oxygen-sensing compound;
(iv) measuring the fluorescence or phosphorescence from the oxygen-sensing compound; and
(v) calculating the concentration of oxygen from the measurement.

21. The method of claim 20, wherein the monitoring is in real-time, in vivo measurement in the tissue.

22. The method of claim 21, wherein the tissue is a deep tissue.

23. The method of claim 22, wherein the deep tissue is selected from the group consisting of heart, lungs, liver, kidneys, and combinations thereof.

24. The method of claim 22, wherein the deep tissue is in and around a valve of a heart.

25. The method of claim 22, wherein the deep tissue is a surgical flap, replanted tissue, or transplanted organ.

26. The method of claim 22, wherein the deep tissue is in and around peripheral arteries.

27. The method of any of claims 17-26, wherein the excitation light source excites at a wavelength in the range of about 620 nm to about 660 nm.

28. A system for monitoring oxygenation, the system comprising (i) an oxygen sensor composition of any of claims 1-15; (ii) an excitation light source; and (iii) an instrument for measuring and reporting fluorescence or phosphorescence from the activated oxygen-sensing compound.

29. The system of claim 28, wherein the monitoring is in real-time, in vivo measurement in the tissue.

30. The system of claim 28, wherein the sensor composition is disposed on a tip of an optic probe.
